(19) United States
(12) Patent Application Publication
(10) Pub. No.: US 2024/0161298 A1
(43) Pub. Date: May 16, 2024

(54) INFORMATION PROCESSING SYSTEM, BIOLOGICAL SAMPLE PROCESSING DEVICE, AND PROGRAM

(71) Applicant: SONY GROUP CORPORATION, TOKYO (JP)

(72) Inventors: SHIORI SASADA, TOKYO (JP); KAZUKI AISAKA, TOKYO (JP); KENJI YAMANE, TOKYO (JP); JUNICHIRO ENOKI, TOKYO (JP); YOSHIYUKI KOBAYASHI, TOKYO (JP); MASATO ISHII, TOKYO (JP); KENJI SUZUKI, TOKYO (JP)

(21) Appl. No.: 18/550,762

(22) PCT Filed: Feb. 7, 2022
(86) PCT No.: PCT/JP2022/004610

§ 371 Date: Sep. 15, 2023

(30) Foreign Application Priority Data
Mar. 29, 2021 (JP) 2021-056228

(51) Int. Cl. G06T 7/00; G16H 50/20

(52) U.S. Cl. CPC G06T 7/0012; G16H 50/20; G06T 2207/10024; G06T 2207/20081; G06T 2207/20084

(57) ABSTRACT

The present invention improves estimation accuracy. An information processing system includes: an acquisition unit (102) configured to acquire adjustment information based on a feature value of learning data used for generation of a learned model that estimates a health condition of a patient or a subject; a processing unit (103) configured to perform processing on a biological sample to be judged on the basis of the adjustment information; and an estimation unit (104) configured to estimate a diagnosis result by inputting measurement data acquired by the processing to the learned model.

### FIELD

The present disclosure relates to an information processing system, a biological sample processing device, and a program.

### BACKGROUND

Recently, a diagnosis support system has been developed, in which a diagnosis by a doctor and the like is supported by outputting a diagnosis estimation result by a learning model based on a medical image which is a pathological image and the like.

### CITATION LIST

### Patent Literature

Patent Literature 1: WO 2020/174863 A

### SUMMARY

### Technical Problem

However, when diagnosis support by Artificial Intelligence (AI) is performed using a digitized medical image, sufficient estimation accuracy (also referred to as a judgment system) may not be obtained if the domain of a learned image is different from that of an image to be used for estimation (hereinafter, also referred to as judgment) of a diagnosis result.

Thus, the present disclosure proposes an information processing system, an information processing device, and an information processing method capable of improving estimation accuracy.

### Solution to Problem

To solve the problems described above, an information processing system according to an embodiment of the present disclosure includes: an acquisition unit configured to acquire adjustment information based on a feature value of learning data used for generation of a learned model that estimates a health condition of a patient or a subject; a processing unit configured to perform processing on a biological sample to be judged on the basis of the adjustment information; and an estimation unit configured to estimate a diagnosis result by inputting measurement data acquired by the processing to the learned model.

### BRIEF DESCRIPTION OF DRAWINGS

FIG. 1 is a schematic diagram illustrating an overall schematic example of a diagnosis support system according to an embodiment.

FIG. 2 is a diagram illustrating an example of analog processes, equipment and drugs to be used in each analog process, and effects expected to be exerted on digital parameters by each analog process.

FIG. 3 is a flowchart illustrating a specific example of a pathological workflow.

FIG. 4 is a diagram illustrating an example of a learning data set and a feature parameter set according to the embodiment.

FIG. 5 is a block diagram illustrating a schematic configuration example of an information processing system according to the embodiment.

FIG. 6 is a diagram illustrating a schematic configuration example of the diagnosis support system according to the embodiment.

FIG. 7 is a block diagram illustrating a configuration example of a derivation device according to the embodiment.

FIG. 8 is a diagram illustrating learning of a learning model according to the embodiment.

FIG. 9 is a diagram illustrating an operation in a case where a diagnosis result is estimated using a learned model according to the embodiment.

FIG. 10 is a diagram illustrating an operation in a case where first preprocessing is executed using a preprocessing model according to the embodiment.

FIG. 11 is a diagram illustrating relearning of a learned model according to the embodiment.

FIG. 12 is a diagram illustrating an example of a user interface for parameter adjustment according to the embodiment.

FIG. 13 is a diagram illustrating another example of the user interface for parameter adjustment according to the embodiment.

FIG. 14 is a diagram illustrating an example of a model management table according to the embodiment.

FIG. 15 is a diagram illustrating an example of a user interface for model selection according to the embodiment.

FIG. 16 is a hardware configuration diagram illustrating an example of a computer that implements a technique according to the present disclosure.

### DESCRIPTION OF EMBODIMENTS

Hereinafter, an embodiment of the present disclosure will be described in detail with reference to the drawings. Note that, in the following embodiment, the same parts are denoted by the same reference signs to omit redundant description.

The present disclosure will be described according to the following order of items.
0. Introduction
1. Embodiment
1.1 General Overview
1.2 Analog Process in Pathological Workflow
1.3 Example of Learning Data Set
1.4 Outline of Information Processing System
1.5 System Configuration
1.6 Derivation Device
1.7 Learning of Learning Model
1.8 Estimation of Diagnosis Result Using Learned Model
1.9 Preprocessing Using Neural Network
1.10 Learning of Preprocessing Model (Transfer Learning)
1.11 Method of Creating/Updating Preprocessing Model
1.11.1 Case Where Learned Model Is White Box
1.11.2 Case Where Learned Model Is Black Box
1.11.3 Others
1.12 Improvement in Estimation Accuracy by Model Selection
1.13 Format Unification
1.14 Example of User Interface for Digital Parameter Adjustment
1.15 Example of User Interface for Model Selection
2. Hardware Configuration

### 0. INTRODUCTION

In order to improve the estimation accuracy of a medical support system, it is necessary to learn a learning model (also referred to as training) while using an enormous amount of medical images as learning data. However, it is difficult to collect a sufficient amount of medical information in one medical facility. Thus, it is conceivable to introduce, into an individual medical facility, a learning model obtained by learning an enormous amount of learning data. However, since medical equipment, its device characteristics and measurement conditions, and the like are different from one medical facility to another, features of learning data used for learning of the learning model and features of measurement data acquired in the individual medical facility are often different, that is, the domain of the learning data and that of the measurement data are often different. For this reason, it is sometimes difficult to estimate a diagnosis result with sufficient accuracy.

To deal with this, in the following embodiment, while focusing on the fact that a medical image such as a pathological slide is digitized through various physical processes such as slicing, staining, and scanning, preprocessing including transfer learning is proposed in which calibration with a difference between these processes reflected as a parameter is performed to enable improvement in estimation accuracy.

In addition, the following embodiment also proposes a configuration for providing feedback to a user. In the configuration, for example, parameters determined by factors such as measurement devices (types, models, and the like) used when medical images are acquired and their device characteristics and measurement conditions (including environmental conditions, setting values, and the like) (hereinafter, these factors are collectively referred to as physical conditions) are classified into parameters that can be adjusted digitally and parameters that cannot be adjusted digitally, and then feedback such that the setting (such as the type of cover glass, the thickness of a pathological section, a drug, and the staining time) of the digital/analog processes of pathology is to be changed according to this classification is provided to a user.

Further, the following embodiment also proposes a configuration for visualizing a range of feature values that can ensure accuracy based on an image used for learning or its features to enable checking as to whether the feature value of judgment target image subjected to conversion falls within this range. In this event, means for presenting the judgment target image subjected to conversion so that a person can check this image may be provided.

Furthermore, in a case where, to cope with an unprecedented disease case, a learning model generated from an image group closest to an image of this disease case is subjected to transfer learning, the following embodiment also proposes a configuration for enabling selection of a learning model to be subjected to transfer learning on the basis of approximation of features between the image of the disease case and each of images of the candidate image group.

### 1. Embodiment

### 1.1 General Overview

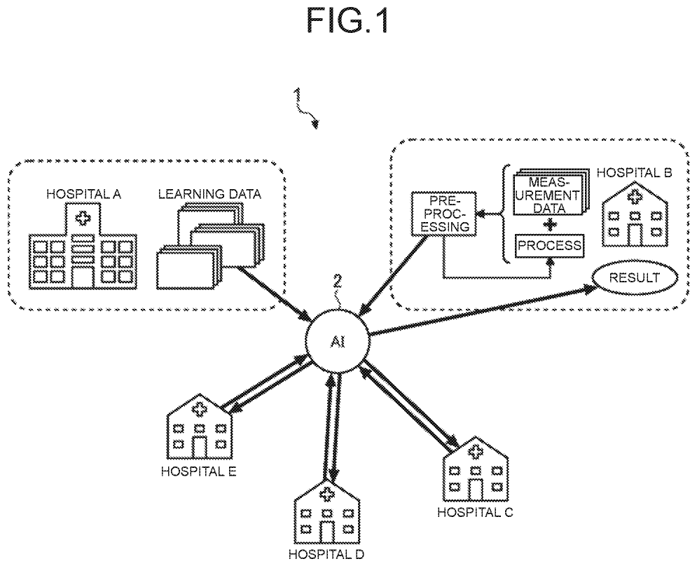

Hereinafter, an overall outline of a diagnosis support system (information processing system, information processing device, and information processing method) according to an embodiment of the present disclosure will be described. FIG. 1 is a schematic diagram illustrating an overall schematic example of the diagnosis support system according to this embodiment.

As illustrated in FIG. 1, in this embodiment, AI2 that estimates information for supporting diagnosis by a user such as a doctor or a pathologist is subjected to learning by using, for example, a set of learning data (hereinafter referred to as a learning data set) collected in a hospital A capable of collecting a sufficient amount of medical information for learning. The learned AI2 is introduced into the other hospitals B to E. Note that, the hospital A may be, for example, a medical facility capable of collecting a large amount of medical information such as a research institution and a university hospital, and the other hospitals B to E may be, for example, various medical facilities such as a research institution, a university hospital, a private hospital, a doctor's office, and a clinic.

In this introduction, in a case where the medical equipment, its device characteristics and measurement conditions, and the like in the hospital A are different from those in the other hospitals B to E, when the AI2 subjected to learning using the learning data set collected in the hospital A is introduced into the other hospitals without change, it is sometimes difficult to estimate a diagnosis result with sufficient accuracy due to a difference between features of learning data collected in the hospital A and features of measurement data to be judged acquired in the other hospitals, that is, a difference in domain between the learning data used for learning and the measurement data to be judged.

Thus, in this embodiment, preprocessing of approximating the features of the measurement data acquired in another hospital (for example, the hospital B) to the features of the learning data set used for learning of the AI2 is executed. Note that, the features of the learning data set may be, for example, a distribution or an average value of feature values (hereinafter also referred to as parameters) of learning data constituting the learning data set.

This embodiment illustrates a case where the learning data and the measurement data are image data obtained by imaging a tissue section taken from a patient. In this case, the features of the learning data and the measurement data may be, for example, the brightness (which may be the color tone), hue, white balance, gamma value, color chart, and the like of the learning data and the measurement data. Note, however, that while not limited to this, the learning data and the measurement data may be various sets of data such as text data and waveform data (including sound data) or mixed data of two or more of these sets of data. For example, in a case where the learning data and the measurement data are text data, their features may be a language type (Japanese, English, etc.), a syntax pattern (habit and the like), a difference in synonyms/quasi-synonyms, and the like.

The preprocessing according to this embodiment can include processing of adjusting (also referred to as correcting) the features of the acquired measurement data (hereinafter referred to as first pre-processing) and processing of adjusting/changing measurement conditions and the like in the process of acquiring the measurement data (also referred to as an analog process in this description) (hereinafter referred to as second preprocessing).

The first preprocessing is processing for domain adaptation which is known as a problem in so-called transfer learning. In the first preprocessing, the features of the digitized measurement data are adjusted directly so as to be approximated to the features of the learning data used for learning of the AI2. This adjustment may be performed automatically or manually. In the case of manual adjustment, for example, a user interface such as a control slide may be provided to a user in order to adjust feature values (hereinafter also referred to as digital parameters) such as brightness (which may be a color tone), hue, white balance, a gamma value, and a color chart. In that case, this user interface may be provided to the user at the stage of inputting the measurement data to the learned AI2, or may be provided to the user at the time of acquiring the measurement data. In other words, the user interface for digital parameter adjustment may be provided to a judgment device that executes judgment based on the measurement data, or may be provided to a measurement device that acquires the measurement data.

Note that, the digital parameters may be various feature values that can be adjusted by digital processing, such as brightness (which may be a color tone), hue, white balance, a gamma value, and a color chart. In addition, in this embodiment in which image data is used as the learning data, the digital parameters may have the same meaning as the features of the learning data and the measurement data described above.

On the other hand, in the second preprocessing, physical conditions (hereinafter referred to as analog parameters) recommended at the time of acquiring the measurement data are specified so that the features of the measurement data acquired by measurement are approximated to the features of the learning data used for learning of the AI2. The specified analog parameters are presented to a user via a display device 24 of a pathological system 20, for example. For example, the user adjusts/changes the measurement conditions and the like according to the analog parameters thus presented. This makes it possible to approximate the domain of the measurement data to the domain of the learning data set at the measurement stage (that is, in the analog process), and thus possible to facilitate transfer learning of the AI2, subjected to learning using the learning data set collected in the hospital A, to the hospitals B to E.

The analog parameters can include various parameters that are manually adjusted by a user in the analog process at the time of acquiring the learning data or the measurement data and, for example, various parameters that cannot be adjusted by digital processing. For example, in a case where the measurement target is a stained tissue section, the various parameters include the type, thickness, and the like of the tissue section, the type, model, manufacturer, and the like of a slicer that slices the tissue section from a block, the type, manufacturer, staining concentration, staining time, and the like of a staining marker that is used for staining, the type, model, manufacture, and the like of a staining machine that is used for staining, the material, thickness, and the like of cover glass that encapsulates the tissue section, the model, manufacturer, gamma value, compression rate, and the like of a camera (also referred to as a scanner) that takes images, the type, manufacturer, output wattage, and the like of an excitation light source. Further, in this embodiment, the analog parameters may include, as parameters, information such as temperature, humidity, and illuminance at the time of acquiring the measurement data, and information such as a technician and a doctor that have acquired the measurement data.

### 1.2 Analog Process in Pathological Workflow

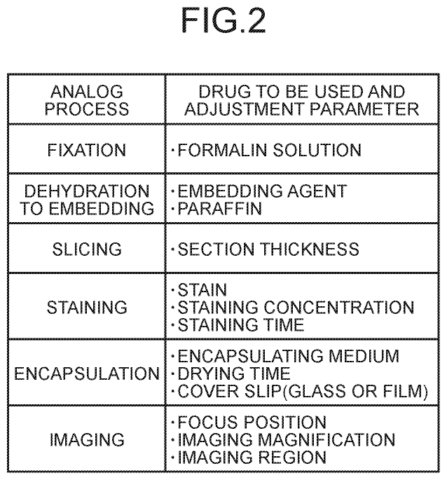

Here, FIG. 2 illustrates a table summarizing an example of the analog processes in a pathological workflow in a case where the measurement target is a tissue section, an example of equipment and drugs used in each analog process, and an example of effects expected to be exerted on the digital parameters by each analog process.

As illustrated in FIG. 2, examples of the analog processes include “fixing”, “dehydration to embedding”, “slicing”, “staining”, “encapsulation”, and “imaging”.

In the “fixation”, for example, a biological block is immersed in a formalin solution to perform a chemical treatment for protecting a biological sample from degradation due to autolysis or decay. This process may indirectly affect the hue of an image obtained by imaging the biological sample.

In the “dehydration to embedding”, the fixed biological block is dehydrated using, for example, an aqueous solution such as ethanol or acetone. Then, the dehydrated biological block is embedded using an embedding agent, such as resin, and paraffin. This process may indirectly affect the hue of an image obtained by imaging the biological sample.

In the “slicing”, for example, a thin section is cut out from the embedded biological block using a microtome and the like. The thickness of the cut thin section can directly affect the brightness of the image obtained by imaging the biological sample. In addition, it may indirectly affect the hue and color of the image.

In the “staining”, the cut thin section is stained using an agent. A stain used for the staining, the staining concentration, and the staining time may affect the hue of the image obtained by imaging the biological sample.

In the “encapsulation”, for example, the stained thin section is placed on slide glass and a cover slip (cover glass or cover film) covers this thin section, thereby preparing a specimen of the thin section. In addition, the thin section specimen covered with the cover slip is dried through a predetermined drying process. The encapsulant, the drying time, the material and thickness of the cover slip, and the like used in this process may affect the brightness, hue, and color of the image obtained by imaging the biological sample.

In the “imaging”, the dried thin section specimen is imaged. In this process, parameters such as a focus position, an imaging magnification, and an imaging region may affect the brightness, hue, and color of the image obtained by imaging the biological sample.

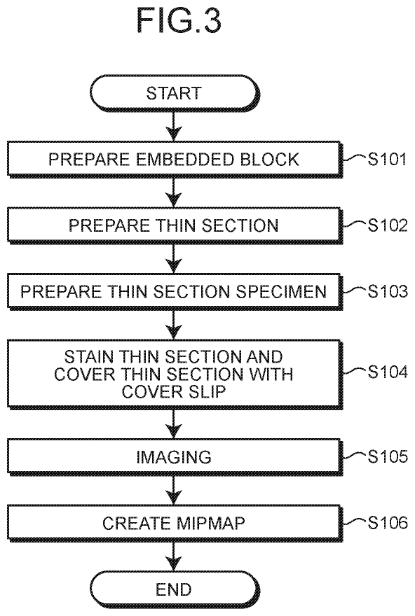

Next, the pathological workflow including the above-described analog processes will be described with specific examples. FIG. 3 is a flowchart illustrating a specific example of the pathological workflow.

As illustrated in FIG. 3, in the pathological workflow, for example, an embedded block is prepared first (step S101). Specifically, a biological sample to be observed is embedded with a hydrophobic embedding agent such as paraffin, that is, the circumference of the biological sample is covered and fixed with this agent.

Subsequently, a thin section is prepared (step S102). Specifically, by using a thin section preparation device, an ultrathin section having a thickness of about 3 to 5 μm is prepared from the embedded block in which the biological sample is embedded.

Next, a thin section specimen is prepared (step S103). Specifically, the thin section prepared by the thin section preparation device is placed on an upper surface of the slide glass, for example, to prepare a thin section specimen used for a physics and chemistry experiment, microscopic observation, and the like.

Next, staining of the thin section specimen and processing of covering the stained thin section with the cover slip are executed (step S104). Various staining methods such as the negative staining method and the mica flake method may be used for staining the thin section specimen. In addition, the process from dyeing to covering with the cover slip may be completed by a series of automatic operations.

Next, after the predetermined drying process, the stained thin section specimen is imaged (step S105). In the imaging, for example, the thin section specimen may be imaged in low resolution and high resolution. For example, the entire thin section specimen is imaged in low resolution, and a region of the thin section present in the specimen is specified from the low resolution image obtained by this imaging. Then, in the high-resolution imaging, the region of the thin section is divided into one or more regions, and each divided region is imaged in high resolution. Note that, high-resolution images acquired by the high-resolution imaging may include a superimposed region used as a margin at the time of stitching.

Next, the acquired high-resolution images are stitched together to create a high-resolution image of the entire thin section (Whole Slide Image (WSI)), and then the resolution of the created WSI is reduced stepwise to generate image data for each layer, thereby creating a mipmap whose resolution changes hierarchically (step S106). Thereafter, this operation ends.

### 1.3 Example of Learning Data Set

The learning data used for learning of the AI2 may be linked with information indicating its features (for example, the digital parameter and/or the analog parameter described above. Such information is hereinafter referred to as a feature parameter). The feature parameter may be so-called metadata, and may be provided to the hospitals B to E as necessary. However, in a case where the feature parameter is not provided to the hospitals B to E or in a case where the feature parameter is not created at the time of learning, a conversion formula for approximating the feature parameter itself or the feature of the measurement data to the feature of the learning data may be generated in the hospitals B to E from the learned AI2 and/or learning data. Note that, the feature parameter may be generated, for example, by analyzing the learning data itself. Meanwhile, for example, the conversion formula may be generated on the basis of a result of actual judgment on the learning data with each of the digital parameters changed.

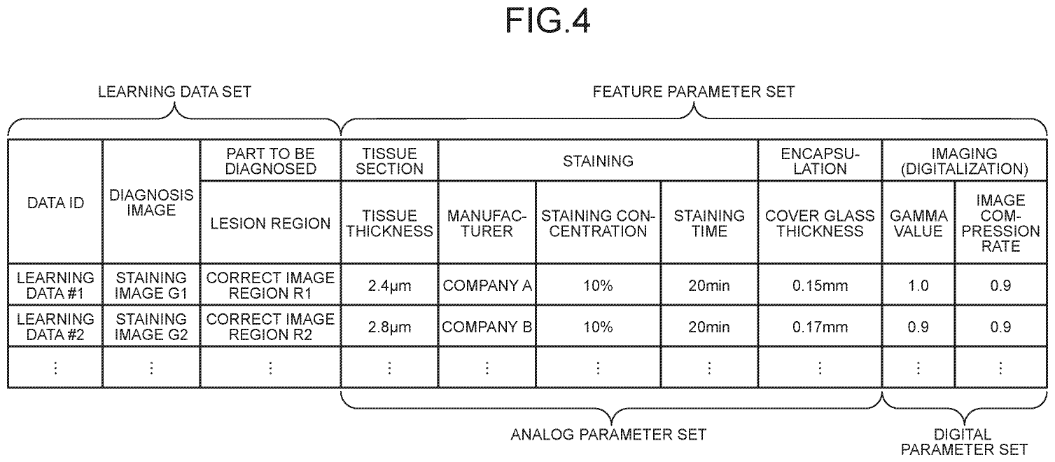

FIG. 4 is a diagram illustrating an example of a learning data set and a feature parameter set according to this embodiment. As illustrated in FIG. 4, the learning data set includes, for example, a set of combinations (corresponding to learning data) of diagnosis images (staining images G1, G2, . . . ) as learning data and lesion regions to be diagnosed (correct region images R1, R2, . . . ) as correct data. For example, each diagnosis image may be assigned a data ID for uniquely identifying the diagnosis image.

In addition, the feature parameter set added to the learning data set includes an analog parameter set and a digital parameter set. The analog parameter set may include, for example, a tissue thickness as a parameter related to a tissue section, a manufacturer of a staining marker as a parameter related to staining, a staining concentration and a staining time, a cover glass thickness as a parameter related to encapsulation of the tissue section, and the like. Meanwhile, the digital parameter may include, for example, a gamma value and an image compression rate as a parameter related to imaging, and the like. However, the present invention is not limited to these, and various modifications may be made including the above-described various parameters. Further, each parameter may be associated with each learning data in the learning data set.

### 1.4 Outline of Information Processing System

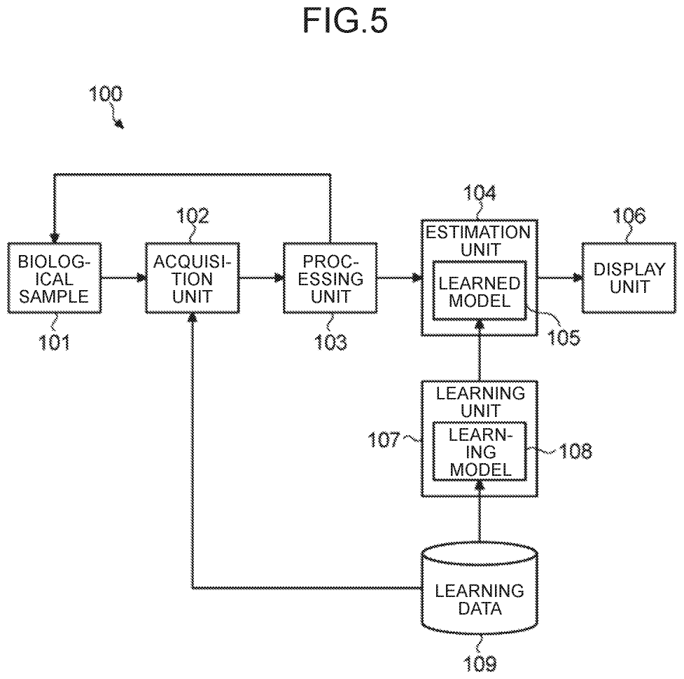

Next, a schematic configuration of the information processing system according to this embodiment will be described with an example. FIG. 5 is a block diagram illustrating a schematic configuration example of an information processing system 100 according to this embodiment. As illustrated in FIG. 5, the information processing system 100 includes: an acquisition unit 102; a processing unit 103; an estimation unit 104; a learning unit 107; and a display unit 106.

The acquisition unit 102 is configured to acquire adjustment information based on the feature values of learning data 109 used to generate a learned model 105 that estimates the health condition of a patient or a subject.

The processing unit 103 is configured to executes predetermined processing on a biological sample 101 to be judged, on the basis of the adjustment information acquired by the acquisition unit 102.

The estimation unit 104 includes the learned model 105, and is configured to input the measurement data acquired by the processing unit 103 to the learned model 105 (the AI2 in FIG. 1, for example) to estimate a diagnosis result.

The learning unit 107 is configured to subject a learning model 108 to training (learning) using the learning data 109 to generate the learned model 105 for estimating a diagnosis result from the measurement data.

The display unit 106 is configured to present the diagnosis result estimated by the estimation unit 104 to a user such as a doctor or a pathologist.

### 1.5 System Configuration

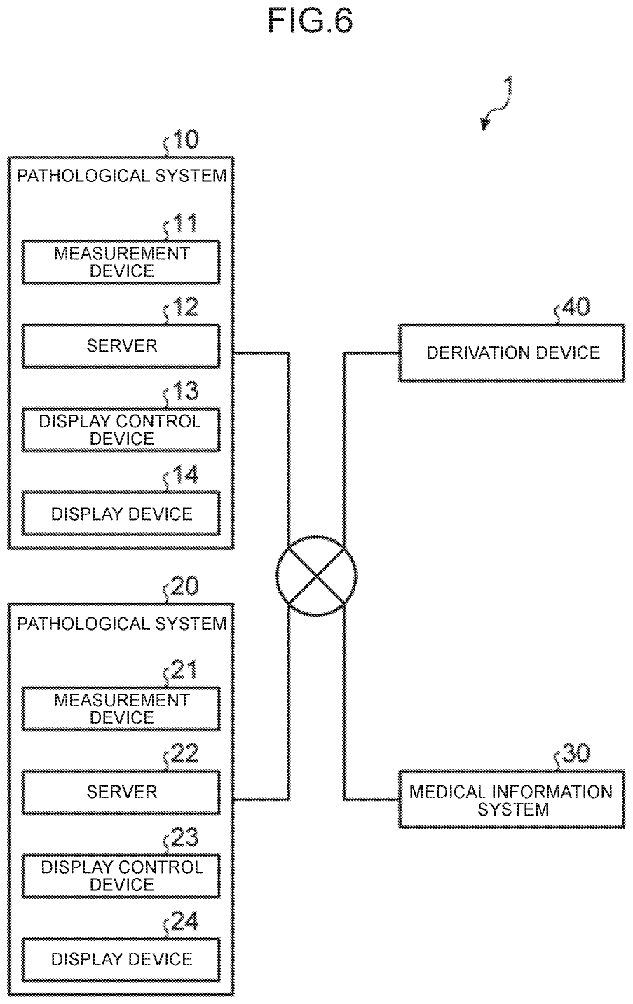

Next, a specific system configuration example used when the information processing system 100 illustrated in FIG. 5 is introduced to a medical site and the like will be described. FIG. 6 is a block diagram illustrating a configuration example of a diagnosis support system (information processing system, information processing device) according to this embodiment. Note that, this description illustrates a case where a learning data set for training the AI2 (corresponding to a learning model to be described later) is acquired from a pathological system 10 and the learned AI2 (corresponding to a learned model 55 to be described later) trained using the learning data set is provided to a pathological system 20. However, while not limited to this, the learning data set may be appropriately modified, for example, the learning data set may be collected from multiple pathological systems such as the pathological systems 10 and 20.

FIG. 6 is a diagram illustrating a schematic configuration example of the diagnosis support system according to this embodiment. As illustrated in FIG. 6, a diagnosis support system 1 includes: the pathological system 10; the pathological system 20; a medical information system 30; and a derivation device 40.

### Pathological System 10/20

The pathological system 10 is a system mainly used by a pathologist, and can correspond to the hospital A in FIG. 1, for example. As illustrated in FIG. 6, the pathological system 10 includes: a measurement device 11; a server 12; a display control device 13; and a display device 14.

The measurement device 11 may be, for example, one or more medical devices and information processing devices that acquire image data of an affected part and image data of a tissue section and the like collected from a patient, such as a Digital Pathology Imaging (DPI) scanner, a Computed Tomography (CT)/Magnetic Resonance Imaging (MRI)/Positron Emission Tomography (PET), a microscope, and an endoscope. Note that, in this example, the image data may be, for example, a stained image of the patient and a tissue section and the like collected from the patient. In this description, the image data may be, for example, a stained image.

In the pathological system 10, the server 12 is configured to provide diagnosis support for a user such as a doctor and a pathologist, and hold and manage image data acquired by the measurement device 11, for example. Note that, the image data acquired by the measurement device 11 may be stored in, for example, a storage unit and the like included in the server 12 or connected to the server.

The display control device 13 is configured to accept, from a user, a request for browsing various kinds of information such as an electronic medical record, a diagnosis result, and an estimated diagnosis result regarding a patient, and send the accepted browsing request to the server 12. The display control device 13 is configured to then control the display device 14 to display the various kinds of information received from the server 12 in response to the browsing request.

The display device 14 has a screen using, for example, liquid crystal, Electro-Luminescence (EL), Cathode Ray Tube (CRT), and the like. The display device 14 may be compatible with 4K or 8K, and may be formed by multiple display devices. The display device 14 can correspond to, for example, the display unit 106 in the configuration illustrated in FIG. 5, and is configured to display various kinds of information to a user according to control performed by the display control device 13.

The pathological system 20 is a system employed in a hospital different from the pathological system 10, and can correspond to the hospitals B to E in FIG. 1, for example. For example, as in the pathological system 10, the pathological system 20 may include: a measurement device 21; a server 22; a display control device 23; and a display device 24. In that case, units included in the pathological system 20 may be the same as those of the pathological system 10, and thus their description will be omitted.

### Medical Information System 30

The medical information system 30 is a so-called electronic medical record system, and is configured to hold and manage information such as a diagnosis result (hereinafter also referred to as diagnosis data) currently or in the past performed on a patient by a doctor, a pathologist, and the like, for example. The diagnosis data is correct data in the learning data, and may be, for example, the lesion regions to be diagnosed (the correct region images R1, R2, . . . ) in FIG. 4. Note that, the diagnosis data may include, for example, identification information (data ID, for example) that links the diagnosis data with the image data. Besides, the diagnosis data may include information for identifying a patient, patient disease information, patient medical history, examination information used for diagnosis, prescription medicine, and the like.

### Derivation Device 40

The derivation device 40 is configured to acquire, for example, image data accumulated every day in the server 12 of the pathological system 10. In addition, the derivation device 40 is configured to acquire diagnosis data accumulated every day in the medical information system 30. The derivation device 40 is configured to generate a learning data set from the collected image data and diagnosis data, and train the learning model using the generated learning data set as teacher data, thereby generating a learned model for estimating a diagnosis result of a patient on the basis of the image data.

Note that, the number of pathological systems included in the diagnosis support system 1 may be three or more. In this case, the derivation device 40 may collect measurement information accumulated in each pathological system, generate a learning data set from the collected image data and diagnosis data, and train the learning model. Also, in the above example, the medical information system 30 may be incorporated in the pathological system 10 and/or 20. In that case, the collected image data and diagnosis data may be stored in the server 12 and/or 22.

Further, the derivation device 40 according to this embodiment may be implemented by a server, a cloud server, and the like arranged on a network, or may be implemented by the server 12/22 arranged in the pathological system 10/20. Alternatively, the derivation device 40 may be implemented in such a way that its parts are arranged in a distributed manner on a system constructed via a network, for example, in such a way that a part of the derivation device is implemented by a server, a cloud server, and the like arranged on a network and the remaining part is implemented by the server 12/22 of the pathological system 10/20.

### 1.6 Derivation Device

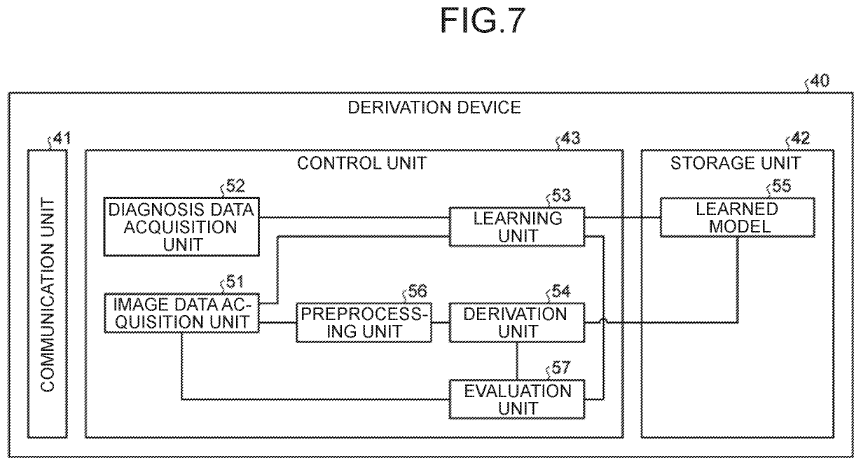

Next, a configuration example of the derivation device 40 according to this embodiment will be described. FIG. 7 is a block diagram illustrating a configuration example of the derivation device according to this embodiment. As illustrated in FIG. 7, the derivation device 40 includes: a communication unit 41; a storage unit 42; and a control unit 43.

The communication unit 41 is implemented by, for example, a Network Interface Card (NIC) and the like. The communication unit 41 is connected to a network (not illustrated) in a wired or wireless manner, and is configured to transmit and receive information to and from the pathological system 10, the pathological system 20, the medical information system 30, and the like via the network. The control unit 43 to be described later is configured to transmit and receive information to and from these devices via the communication unit 41.

The storage unit 42 is implemented by, for example, a semiconductor memory element such as a Random Access Memory (RAM) and a Flash Memory, or a storage device such as a hard disk and an optical disk. The storage unit 42 stores the learned model 55 generated by the control unit 43. The learned model 55 will be described later.

The control unit 43 may be implemented, for example, by causing a Central Processing Unit (CPU) or a Micro Processing Unit (MPU) to execute a program (an example of a diagnosis support program) stored in the derivation device 40 using a Random Access Memory (RAM) and the like as a work area. However, the control unit 43 may be executed by an integrated circuit such as an Application Specific Integrated Circuit (ASIC) and a Field Programmable Gate Array (FPGA).

As illustrated in FIG. 7, the control unit 43 includes: an image data acquisition unit 51; a diagnosis data acquisition unit 52; a learning unit 53; a derivation unit 54; a preprocessing unit 56; and an evaluation unit 57, and is configured to implement or execute functions and actions of information processing described below. Note that, the internal configuration of the control unit 43 is not limited to the configuration illustrated in FIG. 7, and may be another configuration as long as this configuration is for performing information processing to be described later.

The image data acquisition unit 51 is configured to acquire image data, used for training of the learning model performed by the learning unit 53, from the server 12 via the communication unit 41, for example. This image data may be associated with a feature parameter set including an analog parameter set recorded in the analog process and a digital parameter obtained by analyzing the image data. In addition, the image data acquisition unit 51 is configured to acquire image data (corresponding to the measurement data in FIG. 1), used for estimation (judgment) of a diagnosis result performed by the derivation unit 54, from the server 22 via the communication unit 41, for example. The acquired measurement data may be accumulated in the storage unit 42 and the like as appropriate. Note that, in the following description, when the image data used for training of the learning model and the image data used for estimation (judgment) of the diagnosis result are distinguished from each other, the former is referred to as learning image data and the latter is referred to as measurement data.

The diagnosis data acquisition unit 52 is configured to acquire diagnosis data, which is one of the learning data used for training of the learning model performed by the learning unit 53, from the server 12 or the medical information system 30 via the communication unit 41, for example. The acquired diagnosis data may be accumulated in the storage unit 42 and the like as appropriate.

The learning unit 53 can correspond to, for example, the learning unit 107 in the configuration illustrated in FIG. 5, and is configured to generate a teacher data set for training the learning model by linking each piece of the learning image data acquired by the image data acquisition unit 51 with each piece of the diagnosis data acquired by the diagnosis data acquisition unit 52, and train the learning model using the generated learning data set. The learned model 55 thus trained is stored in the storage unit 42, for example, and is appropriately read as needed.

Note that, the method of training the learning model by the learning unit 53 may be based on any algorithm. For example, the learning unit 53 can generate the learned model 55 using various learning algorithms such as deep learning, support vector machine, clustering, and reinforcement learning, which are machine learning methods using a multilayer neural network (Deep Neural Network).

The derivation unit 54 can correspond to, for example, the estimation unit 104 in the configuration illustrated in FIG. 5, and is configured to acquire, when estimation of a diagnosis result for a specific patient is requested from a user via the server 12/22, measurement data of the designated patient and input the measurement data to the learned model 55, thereby causing the learned model 55 to estimate a diagnosis result. The diagnosis result thus estimated is transmitted to the server 12/22, for example, and is displayed on the display device 14/24 under the control of the display control device 13/23.

The preprocessing unit 56 can correspond to, for example, the acquisition unit 102 and the processing unit 103 in the configuration illustrated in FIG. 5, and is configured to execute the first and second preprocessing described above. For example, in the first preprocessing, the preprocessing unit 56 executes preprocessing on the measurement data, acquired from the pathological system 20, so as to approximate the features of the measurement data to the features of the learning image data in the learning data set. The features mentioned here may be digital parameters. For example, the preprocessing unit 56 adjusts each of the digital parameters of the measurement data, such as the brightness (which may be a color tone), hue, white balance, gamma value, and color chart of the measurement data, so that the digital parameter of the measurement data falls within a target range set for the distribution of the corresponding digital parameter in the entire learning image data. Alternatively, the preprocessing unit 56 adjusts each of the digital parameters of the measurement data so as to approximate the digital parameter of the measurement data to a median value (or an average value), a centroid value, and the like in the distribution of the corresponding digital parameter in the entire learning image data. Note that, the target range may be a range of each of the digital parameters for achieving target estimation accuracy, and may be, for example, a range in which the reliability (score, for example) of the estimation result derived by the derivation unit 54 (that is, the learned model 55) is equal to or greater than a preset value or a range in which it is expected to be equal to or greater than this value.

Meanwhile, in the second preprocessing, the preprocessing unit 56 generates information (hereinafter referred to as recommendation information) on analog parameters to be physically adjusted/changed by a user in the measurement stage in order to approximate each of the digital parameters of the measurement data to the corresponding digital parameter of the learning image data, and transmits the generated recommendation information to the server 22. The transmitted recommendation information is displayed on the display device 24 under the control of the display control device 23, for example. On the basis of the recommendation information displayed on the display device 24, the user adjusts/changes the analog parameters such as the type, thickness, and the like of the tissue section, the type, manufacturer, staining concentration, staining time, and the like of the marker that is used for staining, the type, thickness, and the like of the cover glass that encapsulates the tissue section, the model, manufacturer, gamma value, compression rate, and the like of the camera that is used for imaging, the type, manufacturer, output wattage, and the like of the excitation light source, the temperature, humidity, illuminance, and the like at the time of measurement, and a technician and a doctor who perform measurement, thus making it possible to acquire measurement information for judgment having features close to the features of the learning image data. The features mentioned here may be digital parameters.

The evaluation unit 57 is configured to calculate, for example, the reliability (score, for example) of the diagnosis result derived by the derivation unit 54 and evaluate the learned model 55. Note that, the reliability calculated by the evaluation unit 57 may be used for automatic adjustment of digital parameters of the measurement data by the preprocessing unit 56 and/or generation and the like of recommendation information to be provided to a user, as described above.

In addition, the evaluation unit 57 may perform factor analysis on the measurement data in which an error has occurred (for example, measurement data in which the reliability of the diagnosis result is lower than a preset threshold value) to identify a factor (digital parameter in this description) that adversely affects the estimation of the diagnosis result. The factor having an adverse effect may be identified, for example, by classifying the measurement data on the basis of the diagnosis result which is the correct data and the reliability of the derived estimation result, and calculating which digital parameter has strongly contributed to the estimation of the result in this classification using the factor analysis.

In response to this, the preprocessing unit 56 may adjust the digital parameter identified as having an adverse effect. Then, the derivation unit 54 may estimate the diagnosis result again using the measurement data having been subjected to the parameter adjustment.

The digital parameter identified as having an adverse effect may be adjusted automatically or manually. In the case of automatic adjustment, for example, adjustment may be performed in such a way that measurement data in which an error has occurred is identified and, out of digital parameters of this measurement data, a digital parameter having an adverse effect is adjusted so as to fall within a target range or be approximated to a median value or the like.

### 1.7 Learning of Learning Model

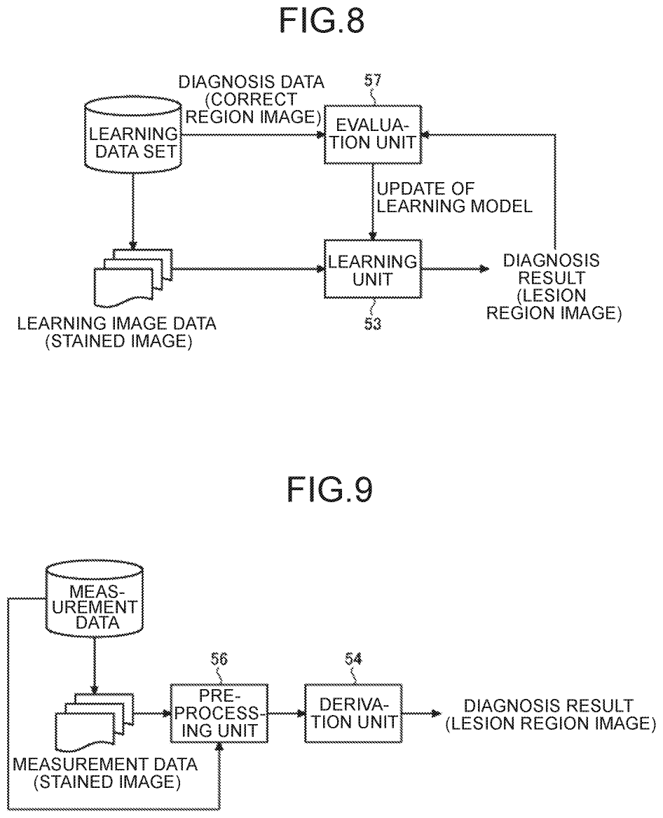

Next, learning of the learning model in the control unit 43 will be described. FIG. 8 is a diagram illustrating learning of the learning model according to this embodiment. As illustrated in FIG. 8, in the learning of the learning model, for example, the learning model before learning stored in the storage unit 42 is read by the learning unit 53. In addition, among the learning data sets including the learning image data acquired by the image data acquisition unit 51 and the diagnosis data acquired by the diagnosis data acquisition unit 52, the learning image data (in this description, a stained image) is input to the learning unit 53. As a result, the diagnosis result (in this description, a lesion region image) derived by the learning model is output from the learning unit 53.

The diagnosis result output from the learning unit 53 is input to the evaluation unit 57. To the evaluation unit 57, diagnosis data in the learning data set (the correct data. In this description, the correct region image) is also input. The evaluation unit 57 evaluates the estimation accuracy of the learning model from the input diagnosis result (estimation result) and diagnosis data (correct data), and updates a hyperparameter of the learning model on the basis of this evaluation result. By iterating such an operation a predetermined number of times or until desired estimation accuracy is obtained, the learned model 55 trained by the learning data set is generated.

### 1.8 Estimation of Diagnosis Result Using Learned Model

Next, a case of estimating a diagnosis result using the learned model 55 generated as described above will be described. FIG. 9 is a diagram illustrating an operation in a case where a diagnosis result is estimated using the learned model according to this embodiment.

As illustrated in FIG. 9, in the estimation of the diagnosis result using the learned model, the preprocessing unit 56 executes the first preprocessing of approximating the features of the learning image data to the features of the one or more pieces of measurement data on the basis of statistical information of the digital parameters in the feature parameter set provided from the source from which the learned model 55 is introduced (for example, the hospital A), and the digital parameters related to the one or more pieces of measurement data collected in the destination to which the learned model is introduced (for example, the hospitals B to E) or their statistical information. In the following description, the feature parameter set linked with the learning data set is referred to as a learning feature parameter set, and the feature parameter or feature parameter set linked with the one or more pieces of measurement data is referred to as a measured feature parameter or a measured feature parameter set.

In this event, for example, the learning feature parameter set linked with the learning data set is input to the preprocessing unit 56. The preprocessing unit 56 may calculate statistical information (for example, a variance value, a median value (or an average value), a centroid value, and the like) of the digital parameters in the learning feature parameter set on the basis of the input learning feature parameter set. However, for example, in a case where the statistical information of the learning feature parameter set is calculated in the hospital A (see FIG. 1), the statistical information held by the hospital A may be input to the preprocessing unit 56 instead of the learning feature parameter set.

In addition, the digital parameters of the measurement data are input to the preprocessing unit 56. However, instead of the digital parameters, the measurement data itself may be input. In that case, the preprocessing unit 56 calculates the value of each digital parameter from the input measurement data. Note that, when multiple pieces of measurement data to be judged exist and a diagnosis result is collectively estimated from the multiple pieces of measurement data, statistical information (for example, a variance value, a median value (or an average value), a centroid value, and the like) of each digital parameter related to the multiple pieces of measurement data may be input to the preprocessing unit 56. Alternatively, the multiple pieces of measurement data themselves or the digital parameters of each piece of measurement data may be input to the preprocessing unit 56. In that case, the preprocessing unit 56 may calculate statistical information (for example, a variance value, a median value (or an average value), a centroid value, and the like) of each digital parameter on the basis of the multiple pieces of measurement data or their digital parameters input to the preprocessing unit.

The preprocessing unit 56 generates a conversion formula for adjusting each digital parameter of the one or more pieces of measurement data from the statistical information of the digital parameter in the learning feature parameter set and the digital parameter or its statistical information related to the one or more pieces of measurement data so that the digital parameter of the one or more pieces of measurement data falls within a target range set for the distribution of the digital parameter in the learning feature parameter set or so that the digital parameter of the one or more pieces of measurement data is approximated to a median value (or an average value), a centroid value, and the like of the distribution of the digital parameter in the learning feature parameter set. For this conversion formula, for example, various formulas such as a simple determinant may be used. Alternatively, the conversion formula may be, for example, a conversion formula that simply replaces each digital parameter of each piece of measurement data with a median value (or an average value) or a centroid value of the distribution of the digital parameter in the learning feature parameter set.

Once generating the conversion formula in this manner, the preprocessing unit 56 adjusts each digital parameter of the one or more pieces of input measurement data (stained image) using the generated conversion formula. As a result, each digital parameter of the one or more pieces of measurement data is adjusted so as to be approximated to the digital parameter of the learning image data in the learning data set. Then, the preprocessing unit 56 outputs, to the derivation unit 54, the measurement data having been subjected to the parameter adjustment.

The measurement data having been subjected to the parameter adjustment and input to the derivation unit 54 is input to the learned model 55 read from the storage unit 42. Thus, since the derivation unit 54 estimates a diagnosis result (lesion region image) on the basis of the measurement data in which each digital parameter is adjusted so as to be approximated to the digital parameter of the learning image data in the learning data set, it is possible to obtain a diagnosis result with higher reliability.

### 1.9 Preprocessing Using Neural Network

The description using FIG. 9 illustrates the case where the preprocessing unit 56 generates the conversion formula from the statistical information of each digital parameter in the learning feature parameter set and the digital parameter or its statistical information related to the one or more pieces of measurement data. However, the preprocessing (first preprocessing) according to this embodiment is not limited to the method of adjusting the digital parameter using the conversion formula. For example, the first preprocessing may be executed using a neural network (hereinafter referred to as a preprocessing model) that accepts inputs of each digital parameter or its statistical information in the learning feature parameter set and the digital parameter or its statistical information related to the one or more pieces of measurement data. As the preprocessing model, various learning models that output image data using image data as an input may be used.

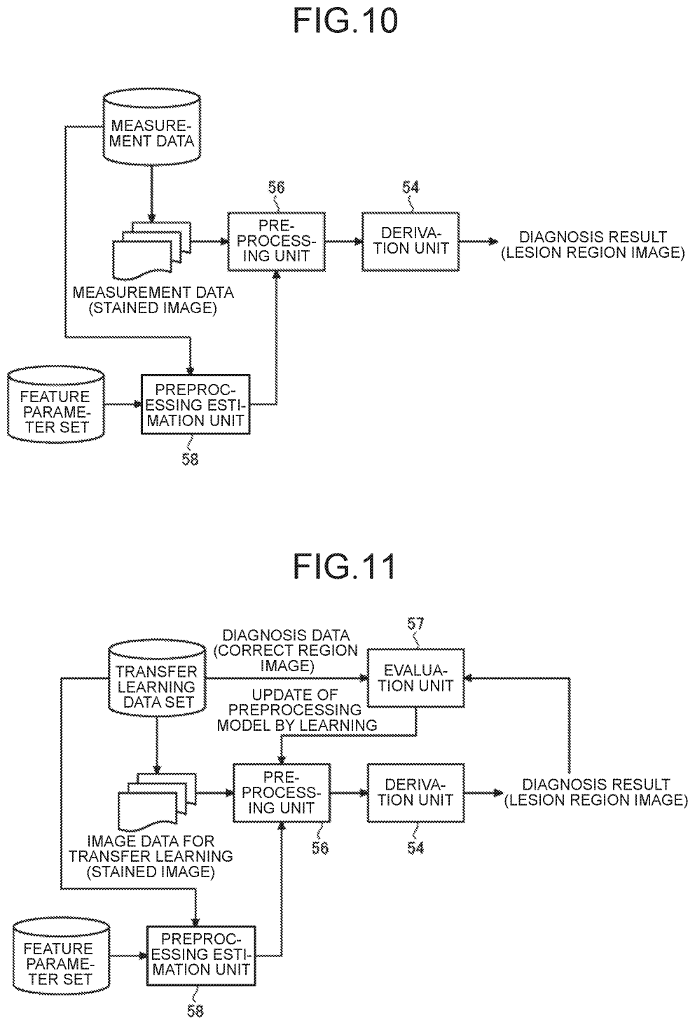

FIG. 10 is a diagram illustrating an operation in a case where the first preprocessing is executed using the preprocessing model according to this embodiment. As illustrated in FIG. 10, in this example, a preprocessing estimation unit 58 for generating a preprocessing model is newly provided.

For example, the learning feature parameter set linked with the learning data set is input to the preprocessing estimation unit 58. The preprocessing estimation unit 58 may calculate statistical information (for example, a variance value, a median value (or an average value), a centroid value, and the like) of the digital parameters in the learning feature parameter set on the basis of the input learning feature parameter set. However, for example, in a case where the statistical information of the learning feature parameter set is calculated in the hospital A (see FIG. 1), the statistical information of the learning feature parameter set held by the hospital A may be input to the preprocessing estimation unit 58 instead of the learning feature parameter set.

In addition, the digital parameters of the measurement data are input to the preprocessing estimation unit 58. However, instead of the digital parameters, the measurement data itself may be input. In that case, the preprocessing estimation unit 58 calculates the value of each digital parameter from the input measurement data. Note that, when multiple pieces of measurement data to be judged exist and a diagnosis result is collectively estimated from the multiple pieces of measurement data, statistical information (for example, a variance value, a median value (or an average value), a centroid value, and the like) of each digital parameter related to the multiple pieces of measurement data may be input to the preprocessing estimation unit 58. Alternatively, the multiple pieces of measurement data themselves or the digital parameters of each piece of measurement data may be input to the preprocessing estimation unit 58. In that case, the preprocessing estimation unit 58 may calculate statistical information (for example, a variance value, a median value (or an average value), a centroid value, and the like) of each digital parameter on the basis of the multiple pieces of measurement data or their digital parameters input to the preprocessing estimation unit.

Upon receiving inputs of the statistical information of each digital parameter in the learning feature parameter set and the digital parameter or its statistical information related to the one or more pieces of measurement data, the preprocessing estimation unit 58 generates a weight parameter w indicating the strength of connection between neurons in the preprocessing model on the basis of the input information. As a result, the preprocessing model is tuned so that each digital parameter or its statistical information related to the one or more pieces of measurement data is approximated to the statistical information of the digital parameter in the learning feature parameter set. Note that, a method of generating the weight parameter w, that is, a method of creating/updating the preprocessing model will be described later.

The preprocessing model tuned by the preprocessing estimation unit 58 is read by the preprocessing unit 56. The preprocessing unit 56 adjusts each digital parameter of the one or more pieces of measurement data (stained image) by inputting the input one or more pieces of measurement data (stained image) to the preprocessing model. As a result, each digital parameter of the one or more pieces of measurement data is adjusted so as to be approximated to the digital parameter of the learning image data in the learning data set. Then, the preprocessing unit 56 outputs, to the derivation unit 54, the measurement data having been subjected to the parameter adjustment.

### 1.10 Learning of Preprocessing Model (Transfer Learning)

Meanwhile, in a case where a certain amount of diagnosed measurement data is accumulated at the introduction destination of the learned model 55 such as the hospitals B to E, the estimation accuracy of the derivation unit 54 can be improved by relearning (transfer learning) the preprocessing model using the measurement data accumulated at this introduction destination. FIG. 11 is a diagram illustrating relearning of the learned model according to this embodiment. Note that, FIG. 11 illustrates a case where FIG. 10 is used as its base. In addition, in FIG. 11, a combination of multiple pieces of accumulated measurement data and diagnosis data (in this example, the correct region image) indicated in diagnosis for each piece of measurement data is illustrated as a transfer learning data set, and each piece of diagnosed measurement data is illustrated as image data for transfer learning.

As illustrated in FIG. 11, in the case of learning the preprocessing model using the image data for transfer learning accumulated in the introduction destination, the diagnosis result output from the derivation unit 54 is input to the evaluation unit 57 in the same configuration as the configuration described using FIG. 10. Note that, the evaluation unit 57 also receives input of diagnosis data (in this example, the correct region image) indicated in diagnosis for each piece of image data for transfer learning.

For example, the evaluation unit 57 evaluates the estimation accuracy of the learned model 55 from the input diagnosis result and diagnosis data (correct region image), and updates the preprocessing model included in the preprocessing unit 56 on the basis of the evaluation result. As a result, since the preprocessing can be optimized so that the estimation accuracy of the derivation unit 54 is improved, the estimation accuracy of the derivation unit 54 can be improved.

### 1.11 Method of Creating/Updating Preprocessing Model

Here, the creation/update of the preprocessing model can be optimized according to the architecture of the learned model 55. However, the creation/update method is different depending on whether the learned model 55 is a white box or a black box, that is, whether the calculation processing inside the learned model 55 is known. Note that, the creation/update of the preprocessing model may be executed by the evaluation unit 57 or may be executed by the preprocessing unit 56.

### 1.11.1 Case Where Learned Model Is White Box

In a case where the learned model 55 is a white box, that is, in a case where calculation processing inside the learned model 55 is exactly known, for example, the preprocessing model may be optimized on the basis of poorness (also referred to as loss) of estimation accuracy of the learned model 55 calculated using a loss function.

For the poorness of estimation accuracy, for example, in a case where a tissue or lesion is identified for each pixel, an average value of identification loss (cross-entropy loss) for each pixel can be used. In addition, in a case where a specific lesion region is detected, a positional deviation error between the detected lesion region and the correct region can be used as the poorness of estimation accuracy.

For optimization of the preprocessing model, each weight parameter w is preferably adjusted so as not to deviate too much from the preprocessing model obtained from the statistical information of the learning feature parameter set. This makes it possible to acquire, as a learning result, the preprocessing model within an appropriate range in consideration of the statistical information of the learning feature parameter set, and avoid estimation of a diagnosis result excessively adapted to a small amount of image data for transfer learning at the introduction destination. For example, assuming that the loss is L and the weight parameter estimated from the learning feature parameter set is w′, an objective function to be minimized in learning can be expressed by the following Formula (1).

L+|w−w′|² (1)

Note that, in a case where the calculation processing inside the learned model 55 is exactly known, the gradient of the objective function can be calculated, and thus the learning of the preprocessing model can be performed similarly to the learning of the normal neural network. For example, by iterating updating of the weight parameter w of the preprocessing model by the stochastic gradient descent method using the calculated gradient, it is possible to learn so as to minimize the loss.

$$w ← w - \frac{d}{dw} ( L + {‖ w - {w}^{′} ‖}^{2} ) \qquad ( 2 )$$

### 1.11.2 Case Where Learned Model Is Black Box

On the other hand, when the learned model 55 is a black box, that is, when the calculation processing inside the learned model 55 is unknown, for example, the preprocessing model may be optimized by searching for the weight parameter w of the preprocessing model that improves the estimation accuracy of the learned model 55.

In the search for the weight parameter w, for example, the weight parameter w that improves the estimation accuracy of the learned model 55 may be searched within a range not more than a certain distance from the weight parameter w′ estimated from the learning feature parameter set. For this search, for example, a method generally used for optimization of a black box function, such as Bayesian optimization or a genetic algorithm (evolutionary computation), may be used.

### 1.11.3 Others

Note that, in the creation/update of the preprocessing model, for example, in a case where there is a digital parameter having a low contribution to the estimation of the diagnosis result, the first preprocessing for this digital parameter (adjustment of the parameter value) may be omitted.

Further, in the above description, the case of effectively executing the transfer learning by generating/updating the weight parameter w of the preprocessing model under the restriction that the weight parameter w is not too far from the preprocessing model estimated from the statistical information of the learning feature parameter set has been illustrated, but the present invention is not limited to this. For example, various changes may be made, including a change such that the distribution of each digital parameter of the measurement data (stained image) is estimated from the statistical information of the learning feature parameter set, and the weight parameter w of the preprocessing model is subjected to learning so that the digital parameter of the measurement data having been subjected to the first preprocessing does not deviate from the estimated distribution.

### 1.12 Improvement in Estimation Accuracy by Model Selection

Meanwhile, for example, in a case where there are multiple learned models 55 and the learned models have been subjected to learning in different domains (that is, different learning data sets), a learned model suitable for the measurement data acquired by the pathological system 20 may be selected from the multiple learned models 55. This selection may be performed manually or automatically. In the case of automatic selection, for example, in a case where there is no learned model having been subjected to learning using the same type of disease as the disease to be inferred, the preprocessing unit 56 may automatically select the learned model having been subjected to learning using the learning data set having features closest to the features of the measurement data acquired from the disease to be inferred among the multiple learned models 55. In that case, the learning unit 53 may relearn the selected learned model with the same kind of disease example. As described above, by automatically selecting the learned model 55 having been subjected to learning in the domain having close features, it is possible to improve the estimation accuracy for a case with a small number of cases.

Meanwhile, for example, in a case where there are multiple preprocessing models and different weight parameters w are set for the respective preprocessing models, the preprocessing model more suitable for the measurement data to be judged, that is, the preprocessing model with improved estimation accuracy of the learned model 55 may be selected. This selection may be performed manually or automatically. In the case of automatic selection, for example, the preprocessing unit 56 or the preprocessing estimation unit 58 may execute the first preprocessing on the measurement data using each of these preprocessing models, and select a more suitable preprocessing model on the basis of evaluation on the diagnosis result obtained when each piece of the measurement data having been subjected to the parameter adjustment obtained by the first preprocessing is input to the learned model 55.

### 1.13 Format Unification

The format of data that can be used as an input is sometimes limited depending on the learning model. For example, most deep learning architectures using a convolutional neural network (CNN) with image data as an input use a square image as an input image. Thus, when an image other than a square image is input, processing of changing the input image data to square image data is generated. In addition, in a case where the acquired digital parameters and analog parameters are different from one medical facility to another, there is a case where the objects to be adjusted in the preprocessing do not match and thus the preprocessing cannot be appropriately executed.

For example, when the sizes of image data acquired in the introduction source (for example, the hospital A) and the introduction destination (for example, the hospitals B to E) are different from each other, the preprocessing unit 56 or the preprocessing estimation unit 58 may convert the size of the image data acquired in the introduction destination into a size suitable for the learned model 55. Further, for example, in a case where digital parameters acquired in the introduction source (for example, the hospital A) and the introduction destination (for example, the hospitals B to E) are different from each other, the preprocessing unit 56 or the preprocessing estimation unit 58 may generate necessary digital parameters from the digital parameters collected in the introduction destination or the measurement data. Note that, regarding the analog parameters, for example, analog parameters to be managed may be presented in advance to the introduction source (for example, the hospital A) and the introduction destination (for example, the hospitals B to E).

### 1.14 Example of User Interface for Digital Parameter Adjustment

Next, a user interface for parameter adjustment provided to a user in a case where the user manually adjusts the digital parameter in the first preprocessing executed by the preprocessing unit 56 will be described with some examples. Note that, the user interface for parameter adjustment may be displayed on the display device 24 by the server 22 via the display control device 23 on the basis of, for example, information transmitted from the preprocessing unit 56 to the pathological system 20.

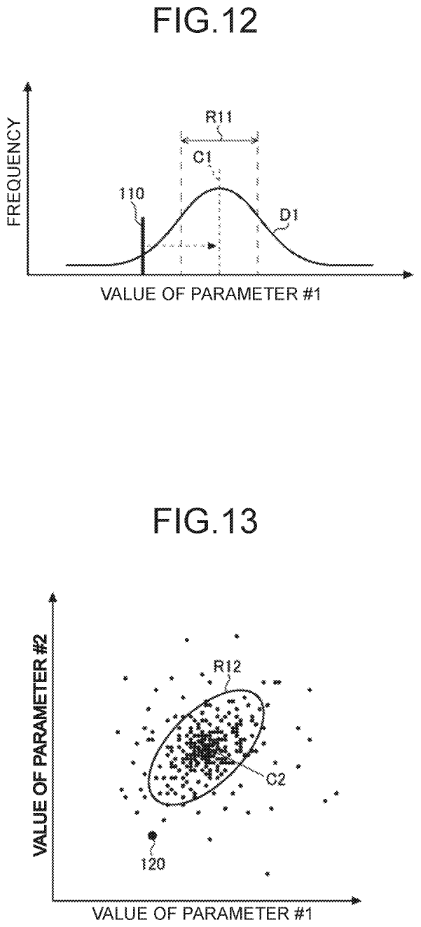

FIG. 12 is a diagram illustrating an example of the user interface for parameter adjustment according to this embodiment. Note that, FIG. 12 illustrates an example of the user interface provided to a user when one parameter #1 (for example, one of brightness (or color tone), hue, white balance, gamma value, color chart, and the like) of the digital parameters of the measurement information for judgment is adjusted.

As illustrated in FIG. 12, in the user interface for parameter adjustment, for example, a graph indicating a distribution D1 of the parameter #1 of the entire measurement information for learning is displayed. In this graph, the horizontal axis may indicate the value of the parameter #1 and the vertical axis may indicate the appearance frequency of each value in the measurement information for learning. However, the present invention is not limited to this, and the vertical axis may be variously changed, for example, the vertical axis may be the accuracy rate (for example, reliability) of the diagnosis result derived at the time of estimation with each value of the parameter #1. Further, this graph may indicate a value range R11 of the parameter #1 in which a desired accuracy rate can be obtained and/or a median value (or an average value) C1 of the distribution of the parameter #1 of the entire measurement information for learning.

For this graph, in the interface for parameter adjustment, a slider 110 indicating the value of the parameter #1 of the measurement information for judgment as an adjustment target is displayed. For example, the slider 110 is movable along the horizontal axis and, in the initial state, indicates the current value of the parameter #1 of the measurement information for judgment as an adjustment target. For example, when a user slides the slider 110 using an input device such as a keyboard, a mouse, or a touch panel, the preprocessing unit 56 adjusts the value of the parameter #1 of the measurement information for judgment as an adjustment target so that the value becomes the adjustment value of the parameter #1 indicated by the slid slider 110. Accordingly, the user can adjust the value of the parameter #1 of the measurement information for judgment as an adjustment target so as to obtain a desired accuracy rate or improve the accuracy rate by moving the slider 110 so that the value is located within the range R11 or approximated to the median value C1.

Meanwhile, FIG. 13 is a diagram illustrating another example of the user interface for parameter adjustment according to this embodiment. Note that, FIG. 13 illustrates an example of the user interface provided to a user when two parameters #1 and #2 (for example, two of brightness (or color tone), hue, white balance, gamma value, color chart, and the like) of the digital parameters of the measurement information for judgment are adjusted.

As illustrated in FIG. 13, in another example of the user interface for parameter adjustment, for example, a graph indicating a two-dimensional distribution D2 of the parameters #1 and #2 of each piece of measurement information in the entire measurement information for learning is displayed. In this graph, the horizontal axis may indicate the value of the parameter #1, and the vertical axis may indicate the value of the parameter #1. Further, this graph may indicate a range R12 of the combination of the parameters #1 and #2 in which a desired accuracy rate can be obtained and/or a centroid value C2 of the distribution of the parameters #1 and #2 of the entire measurement information for learning.

For this graph, in the interface for parameter adjustment, a plot 120 indicating the values of the parameters #1 and #2 of the measurement information for judgment as an adjustment target is displayed. For example, the plot 120 is movable in the two-dimensional coordinate system indicated by the vertical axis and the horizontal axis and, in the initial state, indicates the current values of the parameters #1 and #2 of the measurement information for judgment as an adjustment target. For example, when a user moves the plot 120 using an input device such as a keyboard, a mouse, or a touch panel, the preprocessing unit 56 adjusts the values of the parameters #1 and #2 of the measurement information for judgment as an adjustment target so that the values become the values of the parameters #1 and #2 indicated by the moved plot 120. Accordingly, the user can adjust the values of the parameters #1 and #2 of the measurement information for judgment as an adjustment target so as to obtain a desired accuracy rate or improve the accuracy rate by moving the plot 120 so that the values are located within the range R12 or approximated to the centroid value C2. Note that, the parameters #1 and #2 to be combined may be digital parameters correlated with each other or may be digital parameters having no correlation.

Note that, the above description illustrates the case where the digital parameters of each piece of measurement information for judgment, that is, each piece of image data are manually adjusted using the interface for parameter adjustment. However, the present invention is not limited to this, and the digital parameters of the entire multiple pieces of measurement information for judgment may be collectively adjusted using the interface for parameter adjustment. In this case, the slider 110 illustrated in FIG. 12 or the plot 120 illustrated in FIG. 13 may be an average value, a median value, a centroid value, and the like of the parameter #1 or the parameters #1 and #2 of the entire multiple pieces of measurement information for judgment. Further, each digital parameter of each of the pieces of measurement information for judgment after the adjustment using the slider 110 or the plot 120 may be adjusted so that the spread (for example, a variance value, a full width at half maximum, and the like) of the distribution of the digital parameter of the entire multiple pieces of measurement information for judgment becomes small.

### 1.15 Example of User Interface for Model Selection

Next, a user interface (hereinafter referred to as a user interface for model selection) for causing a user to manually select, in a case where there are multiple learned models 55/preprocessing models, a desired model from the multiple learned models 55/preprocessing models will be described with an example. Note that, in the following description, a configuration for manually selecting the preprocessing model is illustrated, but a configuration for manually selecting the learned model 55 may have the same configuration.

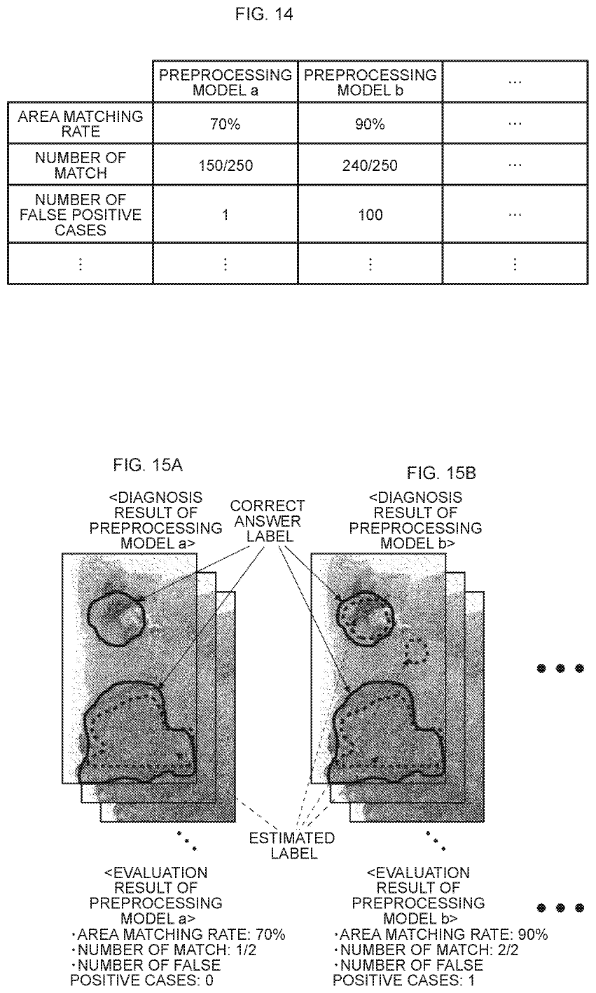

FIG. 14 is a diagram illustrating an example of a model management table according to this embodiment. FIG. 15 is a diagram illustrating an example of the user interface for model selection according to this embodiment.

As illustrated in FIG. 14, in a case where there are multiple candidate preprocessing models (preprocessing models a, b, . . . ) to be read into the preprocessing unit 56, the preprocessing unit 56 or the preprocessing estimation unit 58 may create a model management table that manages an evaluation result by the evaluation unit 57 for a diagnosis result output from the learned model 55 when each of the preprocessing models a, b, . . . is used to execute the first preprocessing. Note that, the evaluation result may be, for example, an area matching rate between a correct answer label (corresponding to a correct answer label (correct answer region image)) attached to the image data for transfer learning by past diagnosis and an estimated label (diagnosis result) estimated by the learned model 55 at the introduction destination, a matching rate between a region where the correct answer label is attached and a region where the estimated label is attached, the number of estimated labels attached to a region other than a region where the correct answer label is attached (number of false positive cases), and the like.

The evaluation result managed in the model management table as described above may be presented to a user via the user interface for model selection together with the diagnosis result for each of the preprocessing models a, b, . . . as illustrated in FIG. 15. In addition, in the user interface for model selection, the measurement data (stained image) used to estimate the diagnosis result and the diagnosis result derived by the learned model 55 (estimated label, corresponding to a lesion region image) may be presented for each of the pre-processing models a, b, . . . Note that, in FIG. 15, a region surrounded by a solid line indicates the correct answer label (corresponding to the correct answer label (correct answer region image)) attached to the image data for transfer learning by past diagnosis, and a region surrounded by a broken line indicates the estimated label (diagnosis result) estimated at the introduction destination. Further, the user interface for model selection may be generated in the preprocessing unit 56 or the preprocessing estimation unit 58 and displayed on the display device 24, or may be generated in the server 22 or the display control device 23 that has received necessary information from the preprocessing unit 56 or the preprocessing estimation unit 58 and displayed on the display device 24.

In the selection of the preprocessing model using the user interface for model selection, a user may select any of displayed images or texts with reference to, for example, the diagnosis result and evaluation result of each preprocessing model displayed on the user interface for model selection. In response to the input of this selection, the preprocessing unit 56 or the preprocessing estimation unit 58 reads the selected preprocessing model or causes the preprocessing unit 56 to read the selected preprocessing model. As a result, the preprocessing unit 56 executes the first preprocessing using the preprocessing model designated by the user.

### 2. Hardware Configuration

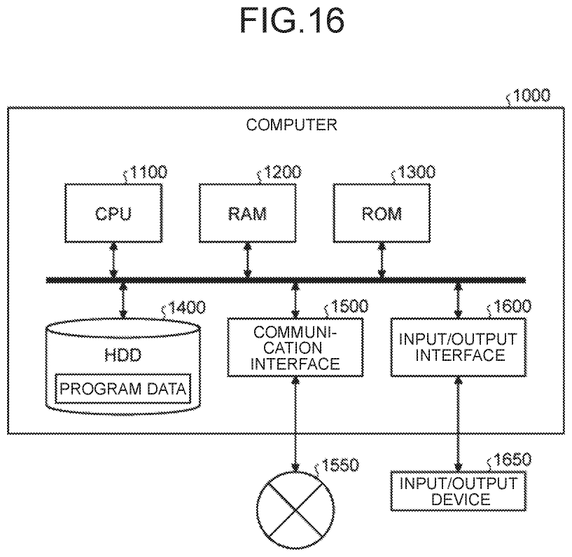

The server 12/22, the display control device 13/23, the medical information system 30, the derivation device 40, and the like according to the embodiment and the modifications described above can be implemented by a computer 1000 having a configuration as illustrated in FIG. 16, for example. FIG. 16 is a hardware configuration diagram illustrating an example of the computer 1000 that implements the functions of the server 12/22, the display control device 13/23, the medical information system 30, the derivation device 40, and the like. The computer 1000 includes a CPU 1100, a RAM 1200, a Read Only Memory (ROM) 1300, a Hard Disk Drive (HDD) 1400, a communication interface 1500, and an input/output interface 1600. These units of the computer 1000 are connected to each other by a bus 1050.

The CPU 1100 operates on the basis of programs stored in the ROM 1300 or the HDD 1400, and controls each unit. For example, the CPU 1100 develops programs stored in the ROM 1300 or the HDD 1400 on the RAM 1200, and executes processing corresponding to the various programs.

The ROM 1300 stores a boot program such as a Basic Input Output System (BIOS) executed by the CPU 1100 when the computer 1000 is activated, a program dependent on hardware of the computer 1000, and the like.

The HDD 1400 is a computer-readable recording medium that non-transiently records a program executed by the CPU 1100, data used by the program, and the like. Specifically, the HDD 1400 is a recording medium that records a program for executing each operation according to the present disclosure which is an example of program data 1450.

The communication interface 1500 is an interface for the computer 1000 to be connected to an external network 1550 (for example, the Internet). For example, the CPU 1100 receives data from another device or transmits data generated by the CPU 1100 to another device via the communication interface 1500.

The input/output interface 1600 has a configuration including the I/F unit 18 described above, and is an interface for connecting an input/output device 1650 and the computer 1000. For example, the CPU 1100 receives data from an input device such as a keyboard and a mouse via the input/output interface 1600. In addition, the CPU 1100 transmits data to an output device such as a display, a speaker, and a printer via the input/output interface 1600. Further, the input/output interface 1600 may function as a media interface that reads a program and the like recorded in a predetermined recording medium (medium). The medium is, for example, an optical recording medium such as a Digital Versatile Disc (DVD) or a Phase change rewritable Disk (PD), a magneto-optical recording medium such as a Magneto-Optical disk (MO), a tape medium, a magnetic recording medium, a semiconductor memory, and the like.

For example, in a case where the computer 1000 functions as the server 12/22, the display control device 13/23, the medical information system 30, the derivation device 40, and the like according to the above-described embodiment, the CPU 1100 of the computer 1000 executes a program loaded on the RAM 1200 to implement the functions of the server 12/22, the display control device 13/23, the medical information system 30, the derivation device 40, and the like. In addition, the HDD 1400 stores a program and the like according to the present disclosure. Note that, the CPU 1100 reads the program data 1450 from the HDD 1400 and executes the program data, but as another example, these programs may be acquired from another device via the external network 1550.

### Others

Among the processes described in the above embodiment, all or a part of the processes described as being automatically performed can be manually performed, or all or a part of the processes described as being manually performed can be automatically performed by a known method. Besides, the processing procedure, specific name, and information including various data and parameters illustrated in the above document and drawings can be arbitrarily changed unless otherwise specified. For example, the various types of information illustrated in the drawings are not limited to the illustrated information.

In addition, each component of each device illustrated in the drawings is functionally conceptual, and is not necessarily physically configured as illustrated in the drawings. In other words, the specific form of distribution and integration of each device is not limited to the illustrated form, and all or a part of the device can be functionally or physically distributed and integrated in an arbitrary unit according to various loads, usage conditions, and the like.

In addition, the above-described embodiment and modifications can be appropriately combined within a range that does not contradict processing contents.

Note that, the effects described in the present specification are merely examples and are not limited, and other effects may be provided.

Note that, the present technique can also have the following configuration.

(1) An information processing system including:
an acquisition unit configured to acquire adjustment information based on a feature value of learning data used for generation of a learned model that estimates a health condition of a patient or a subject;
a processing unit configured to perform processing on a biological sample to be judged on the basis of the adjustment information; and
an estimation unit configured to estimate a diagnosis result by inputting measurement data acquired by the processing to the learned model.

(2) The information processing system according to (1), wherein the learned model is generated using a learning data set including a plurality of pieces of the learning data,
the acquisition unit acquires the adjustment information on the basis of statistical information on a feature value of the plurality of pieces of learning data included in the learning data set, and
the processing by the processing unit is adjustment of a feature value of the measurement data based on the adjustment information.

(3) The information processing system according to (2), wherein
the processing unit adjusts the feature value of the measurement data so that the feature value of the measurement data after adjustment falls within a predetermined range set for the statistical information on the feature value of the plurality of pieces of learning data.

(4) The information processing system according to (3), wherein
the predetermined range is a range in which reliability of an estimation result output from the learned model is equal to or greater than a preset value or a range in which the reliability is expected to be equal to or greater than the value.

(5) The information processing system according to (2), wherein
the processing unit adjusts the feature value of the measurement data so that the feature value of the measurement data after adjustment is approximated to a median value, an average value, or a centroid value in the statistical information on the feature value of the plurality of pieces of learning data.

(6) The information processing system according to any one of (2) to (5), wherein
the processing unit generates, from the statistical information on the feature value of the plurality of pieces of learning data and the feature value of the measurement data, a conversion formula for converting the feature value of the measurement data so that the feature value of the measurement data is approximated to the feature value of the pieces of learning data, and adjusts the feature value of the measurement data using the conversion formula.

(7) The information processing system according to any one of (2) to (5), wherein
the processing unit outputs the measurement data adjusted so that the feature value of the measurement data is approximated to the feature value of the pieces of learning data using a neural network having image data as an input, and
the estimation unit estimates a diagnosis result by inputting the adjusted measurement data to the learned model.

(8) The information processing system according to (7), further including
a preprocessing estimation unit configured to adjust a weight parameter of the neural network on the basis of the statistical information on the feature value of the plurality of pieces of learning data.

(9) The information processing system according to (8), further including
an evaluation unit configured to evaluate the diagnosis result estimated by the learned model and adjust the weight parameter of the neural network on the basis of the evaluation.

(10) The information processing system according to (9), further including
an evaluation unit configured to evaluate the diagnosis result estimated by the learned model, wherein
the processing unit selects one of a plurality of the neural networks, which are different from each other, on the basis of the evaluation output by the evaluation unit for each of the diagnosis results respectively output from the learned model when the plurality of neural networks are used.

(11) The information processing system according to (9), wherein
the processing unit selects one of a plurality of the neural networks on the basis of a neural network selected by a user in a user interface that presents the evaluation output by the evaluation unit for each of the diagnosis results respectively output from the learned model when the plurality of neural networks are used.

(12) The information processing system according to any one of (9) to (11), wherein
the evaluation unit identifies, out of the feature value of the measurement data, a feature value that adversely affects the estimation of the diagnosis result by the learned model, and
the processing unit further adjusts the feature value specified by the evaluation unit.

(13) The information processing system according to (2), wherein
the processing unit adjusts the feature value of the measurement data so that the feature value of the measurement data becomes an adjustment value of the feature value of the measurement data input by a user in a user interface that presents a relationship between the statistical information on the feature value of the plurality of pieces of learning data and the feature value of the measurement data.

(14) The information processing system according to any one of (1) to (13), wherein
the processing unit selects, from a plurality of the learned models having been subjected to learning using different pieces of the learning data, the learned model having been subjected to learning using the learning data having a feature value close to a feature value of the measurement data.

(15) The information processing system according to any one of (1) to (14), wherein
the learning data and the measurement data are image data, and
the feature value includes at least one of brightness, hue, white balance, a gamma value, and a color chart.

(16) The information processing system according to any one of (1) to (15), further including
a display unit configured to present information to a user, wherein
the feature value includes a physical condition at the time of acquiring the learning data,
the processing unit specifies a physical condition at the time of acquiring the measurement data recommended for approximating a feature value of the measurement data to the feature value of the learning data, and
the display unit presents the physical condition specified by the processing unit to the user.

(17) The information processing system according to (16), wherein the physical condition is a parameter manually adjusted by the user in a process of acquiring the learning data or the measurement data.

(18) The information processing system according to any one of (1) to (17), wherein
the learning data and the measurement data are medical images.

(19) A program for causing a computer to function as: an acquisition unit configured to acquire a feature value of learning data used for generation of a learned model that estimates a health condition of a patient or a subject; and an output unit configured to output adjustment information of a processing unit on the basis of a result of comparison between a feature value of measurement data on a biological sample to be judged and a feature value of the learning data.

(20) A biological sample processing device including:
an acquisition unit configured to acquire adjustment information based on a feature value of learning data used for generation of a learned model that estimates a health condition of a patient or a subject; and
a processing unit configured to perform processing on a biological sample to be judged on the basis of the adjustment information.

(21) A program for causing a computer to function as:
an acquisition unit configured to acquire measurement data on a biological sample processed based on a feature value of learning data used for generation of a learned model that estimates a health condition of a patient or a subject; and an estimation unit configured to estimate a diagnosis result by inputting the measurement data to the learned model.

### REFERENCE SIGNS LIST

1 DIAGNOSIS SUPPORT SYSTEM
2 AI
10, 20 PATHOLOGICAL SYSTEM
11, 21 MEASUREMENT DEVICE
12, 22 SERVER
13, 23 DISPLAY CONTROL DEVICE
14, 24 DISPLAY DEVICE
30 MEDICAL INFORMATION SYSTEM
40 Derivation Device
41 COMMUNICATION UNIT
42 STORAGE UNIT
43 CONTROL UNIT
51 IMAGE DATA ACQUISITION UNIT
52 DIAGNOSIS DATA ACQUISITION UNIT
53 LEARNING UNIT
54 DERIVATION UNIT
55 LEARNED MODEL
56 PREPROCESSING UNIT
57 EVALUATION UNIT
58 PREPROCESSING ESTIMATION UNIT
100 INFORMATION PROCESSING SYSTEM
101 BIOLOGICAL SAMPLE
102 ACQUISITION UNIT
103 PROCESSING UNIT
104 ESTIMATION UNIT
105 LEARNED MODEL
106 DISPLAY UNIT
107 LEARNING UNIT
108 LEARNING MODEL
109 LEARNING DATA

1. An information processing system including:
an acquisition unit configured to acquire adjustment information based on a feature value of learning data used for generation of a learned model that estimates a health condition of a patient or a subject;
a processing unit configured to perform processing on a biological sample to be judged on the basis of the adjustment information; and
an estimation unit configured to estimate a diagnosis result by inputting measurement data acquired by the processing to the learned model.

2. The information processing system according to claim 1, wherein
the learned model is generated using a learning data set including a plurality of pieces of the learning data,
the acquisition unit acquires the adjustment information on the basis of statistical information on a feature value of the plurality of pieces of learning data included in the learning data set, and
the processing by the processing unit is adjustment of a feature value of the measurement data based on the adjustment information.

3. The information processing system according to claim 2, wherein
the processing unit adjusts the feature value of the measurement data so that the feature value of the measurement data after adjustment falls within a predetermined range set for the statistical information on the feature value of the plurality of pieces of learning data.

4. The information processing system according to claim 3, wherein
the predetermined range is a range in which reliability of an estimation result output from the learned model is equal to or greater than a preset value or a range in which the reliability is expected to be equal to or greater than the value.

5. The information processing system according to claim 2, wherein
the processing unit adjusts the feature value of the measurement data so that the feature value of the measurement data after adjustment is approximated to a median value, an average value, or a centroid value in the statistical information on the feature value of the plurality of pieces of learning data.

6. The information processing system according to claim 2, wherein
the processing unit generates, from the statistical information on the feature value of the plurality of pieces of learning data and the feature value of the measurement data, a conversion formula for converting the feature value of the measurement data so that the feature value of the measurement data is approximated to the feature value of the pieces of learning data, and adjusts the feature value of the measurement data using the conversion formula.

7. The information processing system according to claim 2, wherein
the processing unit outputs the measurement data adjusted so that the feature value of the measurement data is approximated to the feature value of the pieces of learning data using a neural network having image data as an input, and
the estimation unit estimates a diagnosis result by inputting the adjusted measurement data to the learned model.

8. The information processing system according to claim 7, further including
a preprocessing estimation unit configured to adjust a weight parameter of the neural network on the basis of the statistical information on the feature value of the plurality of pieces of learning data.

9. The information processing system according to claim 8, further including
an evaluation unit configured to evaluate the diagnosis result estimated by the learned model and adjust the weight parameter of the neural network on the basis of the evaluation.

10. The information processing system according to claim 9, further including
an evaluation unit configured to evaluate the diagnosis result estimated by the learned model, wherein
the processing unit selects one of a plurality of the neural networks, which are different from each other, on the basis of the evaluation output by the evaluation unit for each of the diagnosis results respectively output from the learned model when the plurality of neural networks are used.

11. The information processing system according to claim 9, wherein
the processing unit selects one of a plurality of the neural networks on the basis of a neural network selected by a user in a user interface that presents the evaluation output by the evaluation unit for each of the diagnosis results respectively output from the learned model when the plurality of neural networks are used.

12. The information processing system according to claim 9, wherein
the evaluation unit identifies, out of the feature value of the measurement data, a feature value that adversely affects the estimation of the diagnosis result by the learned model, and
the processing unit further adjusts the feature value specified by the evaluation unit.

13. The information processing system according to claim 2, wherein
the processing unit adjusts the feature value of the measurement data so that the feature value of the measurement data becomes an adjustment value of the feature value of the measurement data input by a user in a user interface that presents a relationship between the statistical information on the feature value of the plurality of pieces of learning data and the feature value of the measurement data.

14. The information processing system according to claim 1, wherein
the processing unit selects, from a plurality of the learned models having been subjected to learning using different pieces of the learning data, the learned model having been subjected to learning using the learning data having a feature value close to a feature value of the measurement data.

15. The information processing system according to claim 1, wherein
the learning data and the measurement data are image data, and
the feature value includes at least one of brightness, hue, white balance, a gamma value, and a color chart.

16. The information processing system according to claim 1, further including
a display unit configured to present information to a user, wherein
the feature value includes a physical condition at the time of acquiring the learning data,
the processing unit specifies a physical condition at the time of acquiring the measurement data recommended for approximating a feature value of the measurement data to the feature value of the learning data, and
the display unit presents the physical condition specified by the processing unit to the user.

17. The information processing system according to claim 16, wherein
the physical condition is a parameter manually adjusted by the user in a process of acquiring the learning data or the measurement data.

18. The information processing system according to claim 1, wherein
the learning data and the measurement data are medical images.

19. A program for causing a computer to function as:
an acquisition unit configured to acquire a feature value of learning data used for generation of a learned model that estimates a health condition of a patient or a subject; and
an output unit configured to output adjustment information of a processing unit on the basis of a result of comparison between a feature value of measurement data on a biological sample to be judged and a feature value of the learning data.

20. A biological sample processing device including:
an acquisition unit configured to acquire adjustment information based on a feature value of learning data used for generation of a learned model that estimates a health condition of a patient or a subject; and
a processing unit configured to perform processing on a biological sample to be judged on the basis of the adjustment information.

21. A program for causing a computer to function as:
an acquisition unit configured to acquire measurement data on a biological sample processed based on a feature value of learning data used for generation of a learned model that estimates a health condition of a patient or a subject; and
an estimation unit configured to estimate a diagnosis result by inputting the measurement data to the learned model.
